(19) United States
(12) Patent Application Publication
(10) Pub. No.: US 2007/0191803 A1
(43) Pub. Date: Aug. 16, 2007

(54) Binder compositions

(76) Inventors: David Diehl, Akron, OH (US); Theodore DelDonno, Holden, MA (US); Carla Dittman McBain, Wadsworth, OH (US); David Lee, Rock Hill, SC (US); Robert Holmes, Akron, OH (US); William Sands, Akron, OH (US)

Correspondence Address: Chief Intellectual Property Counsel;Omnova Solutions Inc, 175 Ghent Road, Fairlawn, OH, 44333, US

(21) Appl. No.: 10/586,197

(22) PCT Filed: Jan. 14, 2005
(86) PCT No.: PCT/US05/01168

§ 371 Date: Jul. 14, 2006

Related U.S. Application Data
(60) Provisional application No. 60/537,592, filed on Jan. 16, 2004.
(60) Provisional application No. 60/581,732, filed on Jun. 22, 2004.

(51) Int. Cl. A61F 13/15

(52) U.S. Cl. 604/385.17

(57) ABSTRACT

A binder composition for a non-woven fabric, the binder composition comprising a latex, the latex including a heterogeneous blend of dispersed polymer particles and a surfactant, where the particles include from about 55 to about 100% by weight of a gelled polymer, from about 1 to about 15% by weight polymeric units bearing an acid functionality, and where dried films of the latex exhibit a Tg of from about −50° C. to about 60° C.

This application claims the benefit of International Application No. PCT/US2005/001168, filed Jan. 14, 2005, which claims the benefit of U.S. Provisional Application Ser. No. 60/537,592, filed on Jan. 16, 2004, and U.S. Provisional Application Ser. No. 60/581,732, filed on Jun. 22, 2004, which are incorporated herein by reference.

### FIELD OF THE INVENTION

This invention relates to binder compositions; in one embodiment, these compositions are useful for non-woven fabrics especially permeable layer of personal hygiene articles.

### BACKGROUND OF THE INVENTION

A non-woven fabric is a web or continuous sheet of fibers laid down mechanically. The fibers may be deposited in a random manner or oriented in one direction. Most widely used fibers include cellulosics, polyamides, polyesters, polypropylene and polyethylene. Spun fibers, which may be drawn, are laid down directly onto a belt by carding, airlaying or wet-laying.

The continuous sheet can be bonded together with a latex binder and subsequently treated in an oven or a calendar to complete the bonding process. Commonly used lattices for non-woven fabrics may include polymers of butadiene-styrene, butadiene-acrylonitrile, vinyl acetate, and acrylic monomers such as methyl acrylate, ethyl acrylate, methyl methacrylate. When used to bind a non-woven fabric, such as a permeable sublayer of a personal hygiene article, the latex binder desirably possess adequate tensile strength, a high modulus or stiffness under certain conditions, and good textile qualities such as tenacity and hand.

### SUMMARY OF THE INVENTION

One or more embodiments of the present invention provides a binder composition for a non-woven fabric, the binder composition comprising a latex, the latex including a heterogeneous blend of dispersed polymer particles and a surfactant, where the particles include from about 55 to about 100% by weight of a gelled polymer, from about 1 to about 15% by weight polymeric units bearing an acid functionality, and where dried films of the latex exhibit a Tg of from about −50° C. to about 60° C.

One or more embodiments of the present invention also provides a non-woven fabric bound with a binder composition, the binder composition comprising a latex, the latex including a heterogeneous blend of dispersed polymer particles and a surfactant, where the particles include from about 55 to about 100% by weight of a gelled polymer, from about 1 to about 15% by weight polymeric units bearing an acid functionality, and where dried films of the latex exhibit a Tg of from about −50° C. to about 60° C.

One or more embodiments of the present invention further provides a diaper sub-layer comprising a non-woven fabric bound with a binder composition, the binder composition comprising a latex, the latex including a heterogeneous blend of dispersed polymer particles and a surfactant, where the particles include from about 55 to about 100% by weight of a gelled polymer, from about 1 to about 15% by weight polymeric units bearing an acid functionality, and where dried films of the latex exhibit a Tg of from about −50° C. to about 60° C.

### DETAILED DESCRIPTION OF ILLUSTRATIVE EMBODIMENTS

The binder compositions are aqueous emulsions or latexes including a heterogeneous blend of dispersed polymer particles. The polymer particles include one or more polymers, which may be the same or different. In certain embodiments, these binder compositions preferably include at least one anionic surfactant and optionally at least one surface-active agent.

The polymer particles are characterized by having a Tg from about −50° C. to about 60° C., preferably from about −35° C. to about 35° C., and more preferably from about −25° C. to about 25° C., where the Tg is typically determined based upon dried samples or films of the latex using DSC techniques.

The polymer particles are also characterized by having a gel content of from about 55 to about 100%, preferably from about 75 to about 98%, and more preferably from about 80 to about 95% based upon the entire weight of the particles, where gel is determined based on insoluble fractions within a solvent such as THF or toluene.

Further, the polymer particles include one or more polymers characterized by including from about 1 to about 15%, preferably from about 4 to about 12%, and more preferably from about 6 to about 11%, units bearing an acid functionality, i.e., a carboxylic acid group based upon the entire weight of the polymer particles. Acid content can be determined based upon the weight of the acid bearing monomers employed in synthesizing the polymer or by FTIR techniques.

The polymer particles include at one or more polymers having monomeric units deriving from at least one soft monomer, at least one acidic monomer, and optionally at least one hard non-acidic monomer.

Soft monomers include those that upon polymerization (i.e., homopolymerization) give rise to elastomeric polymers or polymers having a Tg below about 0° C., preferably below about −35° C., and more preferably below about −55° C. Useful soft monomers include conjugated dienes, butyl acrylates, 2-ethyl hexylacrylate, hydroxyethylacrylate, dimethacrylates, polyethylene glycol diacrylates, alkyl acrylates, vinyl versatate derived monomers, and mixtures thereof. Exemplary conjugated dienes include, but are not limited, 1,3-butadiene, isoprene, 1,3-pentadiene, 1,3-hexadiene, 2,3-dimethyl-1,3-butadiene, 2-ethyl-1,3-butadiene, 2-methyl-1,3-pentadiene, 3-methyl-1,3-pentadiene, 4-methyl-1,3-pentadiene, and 2,4-hexadiene.

Acidic monomers include those monomers that include both a carboxylic acid group as well as a polymerizable group. Acidic monomers can include both hard and soft monomers. Useful acidic monomers include α,β-unsaturated carboxylic acids, vinyl versatic acids, and mixtures thereof. Exemplary, α,β-unsaturated carboxylic acids include, but are not limited to, methacrylic acid, itaconic acid, citraconic acid, cinnamic acid, acrylic acid, fumaric acid, maleic acid, acids derived form anhydrides such as maleic anhydride, and mixtures thereof.

The hard non-acidic monomers include those monomers that do not include a carboxylic acid functionality and that upon polymerization give rise to thermoplastic polymers or those polymers having a Tg in excess of about 0° C., preferably in excess of about 75° C., and more preferably in excess of about 90° C. Useful hard non-acidic monomers include vinyl aromatic monomers such as styrene, α-methyl styrene, t-butyl styrene, alkyl substituted styrene, divinyl benzene, and mixtures thereof, as well as polyunsaturated divinyl compounds. Other useful hard non-acidic monomers include acrylates such as methyl methacrylate, butyl methacrylate, vinyl acetate, and mixtures thereof. Still other useful hard non-acidic monomers include acrylamides such as methyl acrylamide, 2-acrylamido-2-methylpropane sulfonic acid, the salts of this acid (e.g., sodium, potassium, or ammonium salts), and mixtures thereof.

As is known in the art, the relative amounts of the various monomers employed to synthesize the polymer may be tailored, in order to achieve the desired polymer characteristics set forth above. Also, especially in the case of the gel content, the degree of gel can be controlled by manipulating the conversion time, the polymerization temperature, and the type and level of chain transfer agent.

In one embodiment, the polymer particles preferably include from about 75 to about 15% by weight, preferably from about 65 to about 25% by weight, and more preferably from about 60 to about 35% by weight units deriving from soft monomer, based upon the entire weight of the particle.

In order to achieve the desired Tg level, the polymer particles may preferably include from about 15 to about 75% by weight, preferably from about 25 to about 65% by weight, and even more preferably from about 35 to about 60% by weight hard non-acidic monomer, based upon the entire weight of the particle, units deriving from hard non-acidic monomer. Those skilled in the art will appreciate the level of hard monomer employed in synthesizing the polymer is directly related to the Tg of the polymer. In other words, as the level of hard monomer is increased, the Tg will increase.

In one particularly preferred embodiment, the polymer particles include copolymer synthesized from monomers including 1,3-butadiene, styrene, methacrylic acid, acrylic acid, and optionally itaconic acid. Preferably, the polymer particles include from about 35 to about 70% by weight monomeric units deriving from 1,3-butadiene, from about 15 to about 75% by weight monomeric units deriving from styrene, from about 0 to about 8% by weight monomeric units deriving from methacrylic acid, and from about 0 to about 8% by weight monomeric units deriving from acrylic acid, based on the entire weight of the particle. More preferably, the polymer particles include from about 40 to about 65% by weight monomeric units deriving from 1,3-butadiene, from about 25 to about 65% by weight monomeric units deriving from styrene, from about 1 to about 7% by weight monomeric units deriving from methacrylic acid, and from about 1 to about 7% by weight monomeric units deriving from acrylic acid, based on the entire weight of the particle. Even more preferably, the polymer particles include from about 45 to about 60% by weight monomeric units deriving from 1,3-butadiene, from about 35 to about 45% by weight monomeric units deriving from styrene, from about 2 to about 6% by weight monomeric units deriving from monomeric units deriving from methacrylic acid, from about 0.5 to about 3% by weight monomeric units deriving from deriving from itaconic acid, and from about 2 to about 6% by weight monomeric units deriving from acrylic acid, based on the entire weight of the particle. Still more preferably, the polymer particles include from about 48 to about 58% by weight monomeric units deriving from 1,3-butadiene, from about 37 to about 43% by weight monomeric units deriving from styrene, from about 2.5 to about 5.0% by weight monomeric units deriving from monomeric units deriving from methacrylic acid, from about 1 to about 2% by weight monomeric units deriving from deriving from itaconic acid, and from about 2.5 to about 5.0% by weight monomeric units deriving from acrylic acid, based on the entire weight of the particle.

The binder compositions of this invention include an aqueous emulsion or latex of the polymer particles disclosed above. Additionally, this composition preferably includes a surfactant.

In one embodiment, the surfactant includes an alkali metal salt of an alkyl sulfosuccinate. Useful alkali salts of alkyl sulfosuccinates include sodium dihexyl sulfosuccinate, sodium dioctyl sulfosuccinate, sodium octane sulfonate, alkyl phenol ethoxylates, fatty alcohol ethoxylates, alkyl polyglucosides, alkyl phosphates, and mixtures thereof. Useful surfactants include those available under the tradenames Aerosol™ MA-80 (Cytec), Gemtex™ 80 (Finetex), or MM-80™ (Uniqema).

In another embodiment, the surfactant includes salts of alkyl sulfates and salts of organo disulfonates. Useful salts of alkyl sulfates include sodium lauryl sulfate, which is available under the tradename Stepanol WA as well as Texapon™ (Cognis), Polystep™ B-3 (Stepan), Polystep™ B-5 (Stepan), or Rhodapon™ UB (Rhodia). Useful salts of organo disulfonates include sodium dodecyl diphenyloxide disulfonate, which is available under the tradename Dowfax 2A1 as well as Stepanol™ AM, Polystep™ B-7 (Stepan), Rhodapon™ L-22EP (Rhodia), Dowfax™ 2A1 (Dow), Calfax™ DB-45 (Pilot), Rhodacal™ DSB(Rhodia), or Aerosol™ DPOS-45 (Cytec). Other useful surfactants include sodium laureth sulfate, Laureth-3 (a.k.a. triethylene glycol dodecyl ether), Laureth-4 (a.k.a. PEG-4 lauryl ether), Laureth-5 (a.k.a. PEG-5 lauryl ether), Laureth-6 (a.k.a. PEG-6 lauryl ether), Laureth-7 (a.k.a. PEG-7 lauryl ether), sodium lauryl ether sulfate, sodium laureth-12 sulfate (a.k.a PEG (12) lauryl ether sulfate, and sodium laureth-30 sulfate (a.k.a. PEG (30) lauryl ether sulfate). Other ether alkyl sulfates are available under the tradenames Polystep™ B40(Stepan) or Genapol™ TSM.

In yet another embodiment, especially where the binder composition is foamed, the composition may include a froth agent such as disodium stearyl sulfosuccinamate, which is available under the tradenames Aerosol™ 18, Aerosol™ A18P (Cytec), Monawet™ SNO (Uniqema), Octosol™ 18 (Tiarco), Stanfax™ 318, 319, 377 (Para-Chem). These surfactants (froth agents) may be employed in conjunction with one or more of the surfactants described above or together with thickeners such as sodium carboxymethylcellulose.

The surfactant is typically present within the binder composition in an amount from about 0.1 to about 10% by weight, preferably from about 1 to about 6% by weight, even more preferably from about 2 to about 4% by weight based upon the total weight of the composition. Stated another way, the surfactant is present in an amount from about 0.2 to about 1.0, preferably from about 0.25 to about 0.65, preferably from about 0.35 to about 0.55, more preferably from about 0.40 to about 0.50, and still more preferably from about 0.44 to about 0.48 parts by weight surfactant per 100 parts by weight polymer, where the parts by weight surfactant refer to active surfactant content.

The emulsion polymer of this invention is preferably prepared by employing conventional emulsion polymerization techniques such as those described in U.S. Pat. Nos. 5,166,259 and 6,425,978, which are incorporated herein by reference. In general, these processes employ the use of a free-radical initiator to initiate the polymerization of monomer in the presence of a surfactant. Advantageously, this polymerization takes place in the presence of the surfactant that is preferably present in the binder composition.

Preparation of the polymers or latexes of this invention is not limited to any particular emulsion polymerization technique. Accordingly, a single-charge batch polymerization process may be used, a continuous system may be used, which typically employs a CSTR, a semi-batch or continuous-feed process may be used, or an incremental process may be employed.

Polymerization is typically carried out at a temperature of about 60° C. to about 90° C., and preferably from about 65 to about 80° C.

Any of those free-radical emulsion polymerization initiators conventionally employed in the art may be employed in preparing the polymers or emulsion latexes of this invention. Exemplary initiators include ammonium persulfate, sodium persulfate, potassium persulfate, tert-butyl hydroperoxide, and di-tert-butyl cumene. These initiators may be used in conjunction with a reducing agent such as iron salts, amines, ascorbic acids, sodium salts of ascorbates, sodium formaldehyde sulfoxylates, and mixtures thereof. Conventional amounts of initiator and reducing agent can be used in preparing the latexes of this invention. For example, in one embodiment, about 0.05 to about 2.5, and preferably from about 0.1 to about to about 2.0 parts by weight initiator per 100 parts by weight monomer is used.

The surfactant employed during the polymerization process may include any of those surfactants conventionally employed in the art. As noted above, the preferred surfactant includes those that are also useful for the binder composition. In addition to those described above, other surfactants that may be used (in addition to or in lieu of those described) include alkyl sulfates, alkyl sulfosuccinates, alkyl aryl sulfonates, a-olefin sulfonates, fatty or rosin acids salts, NPE, alkyl aryl sulfonates, alkyl phenol ethoxylates, fatty acid alcohol ethoxylates, and mixtures thereof.

Conventional amounts of the surfactant can be used in synthesizing the latex, although it may be preferred to employ the type and amount of surfactant used in the binder composition.

In one embodiment, the surfactant includes a blend of sodium dihexyl sulfosuccinate and sodium dioctyl sulfosuccinate. The blend can be adjusted to control or obtain a desired critical micelle concentration. The dihexyl to dioctyl weight ratio can vary from 0.05:1 to 1:0.05.

Any of those chain transfer agents conventionally employed in the emulsion polymerization of conjugated diene monomers may be employed in preparing the polymers or latexes of this invention. Exemplary chain transfer agents include, alkyl mercaptans, carbon tetrachloride, carbon tetrabromide, C₂-C₂₂ n-alkyl alcohols, C₂-C₂₂ branched alcohols, 2,4-diphenyl-4-methyl-1-pentene, and mixtures thereof.

Conventional amounts of the chain transfer agent can be used in synthesizing the latex, although it may be preferred to employ the type and amount of chain transfer agent used in the binder composition.

The pH of the latex can be neutralized by the addition of a base such as potassium hydroxide, sodium bicarbonate, ammonium hydroxide, sodium hydroxide, organic amines such as triethylamine, AMP 95, and mixtures thereof.

As those skilled in the art will appreciate, it is conventional to neutralize the latex to a pH of about 4.5 to about 8.0, and preferably from about 5.5 to about 7.5.

In one preferred method, the polymers or emulsions utilized in the present invention are prepared by employing an incremental polymerization technique. Even more preferably, the method includes the use of a polymer seed such as one prepared by the polymerization of itaconic acid and styrene in the presence of a suitable surfactant. Once the seeds are prepared, incremental additions of butadiene monomer, styrene monomer, and acrylic and methacrylic acid monomer, initiator, chain transfer agent, and surfactant are introduced. A similar technique is set forth in U.S. Pat. No. 6,425,978, which is incorporated herein by reference. In one embodiment, the polymers are prepared by polymerizing monomer including from about 35 to about 70% by weight 1,3-butadiene, from about 15 to about 75% by weight styrene, from about 0 to about 8% by weight methacrylic acid, and from about 0 to about 8% by weight acrylic acid, based on the entire weight of the monomer. In other embodiments, the monomer includes from about 40 to about 65% by weight 1,3-butadiene, from about 25 to about 65% by weight styrene, from about 1 to about 7% by weight methacrylic acid, and from about 1 to about 7% by weight acrylic acid, based on the entire weight of the monomer. In other embodiments, the monomer includes from about 45 to about 60% by weight 1,3-butadiene, from about 35 to about 45% by weight styrene, from about 2 to about 6% by weight methacrylic acid, from about 0.5 to about 3% by weight itaconic acid, and from about 2 to about 6% by weight acrylic acid, based on the entire weight of the monomer. In other embodiments, the monomer includes from about 48 to about 58% by weight 1,3-butadiene, from about 37 to about 43% by weight styrene, from about 2.5 to about 5.0% by weight methacrylic acid, from about 1 to about 2% by weight itaconic acid, and from about 2.5 to about 5.0% by weight acrylic acid, based on the entire weight of the monomer.

Depending on the polymerization technique employed, and more specifically the type and quantity of surfactant employed, the latex resulting from the polymerization discussed above can be employed as the binder composition. Alternatively, surfactant can be post added to the latex after polymerization. Likewise, the surface-active agents can be post added after polymerization.

In one embodiment, the polymer is polymerized in the presence of an alkali metal salt of an alkyl sulfate (e.g. sodium lauryl sulfate), and an alkali metal salt of an organodisulfonate (e.g. sodium dodecyl diphenyloxide disulfonate) is post added.

The binder compositions of this invention are particularly useful for binding non-woven fabrics. In one embodiment, the binder can be used to bind the permeable sub-layers of personal hygiene articles such as diapers and feminine hygiene articles.

The permeable sub-layer of the personal hygiene article is a web or mat comprised of randomly arranged non-woven fibers having an open structure and high loft. The web may be formed by carding when the fibers have an appropriate length and flexibility. During carding, the crimped fibers are placed on a moving support and then treated with the binder composition.

The fibers may include natural textile fibers such as jute, sisal, ramie, hemp, and cotton as well as many artificial organic textile fibers or filaments including rayon, those of cellulose esters such as cellulose acetate, vinyl resin fibers such as those of polyvinyl chloride, copolymers of vinyl chloride with vinyl acetate, vinylidene chloride or acrylonitrile, copolymers of acrylonitrile with vinyl chloride, vinyl acetate, methacrylonitrile, vinyl pyridine, polymers and copolymers of olefins such as ethylene and propylene, also condensation polymers such as polyamides or nylon types, polyesters such as ethylene glycol terephthalate polymers and the like.

The fibers may be of one composition or mixtures of fibers in a given web. The preferred fibers include polyolefins, especially polyesters, poly(ethylene terephthalate), acrylics, polyamides, and polypropylene. The polyolefin fibers may include polypropylene, polyethylene, polybutene and their copolymers. The polyester fibers may include any long chain synthetic polymer composed of at least 85% by weight of an ester of a dihydric alcohol and terephthalic acid such as polyethylene terephthalate, and, in addition liquid crystal polyesters, thermotropic polyesters and the like. The acrylic fibers include any fiber forming substance containing a long chain synthetic polymer composed of at least 85% by weight acrylonitrile units —CH₂CH(CN)—. Other types of fibers may also be employed such as high modulus fibers more commonly known as graphite fibers made from rayon, polyacrylonitrile or petroleum pitch.

The fibers may be of most any suitable size and randomly arranged to most any suitable thickness depending upon the desired end use of the non-woven fabric. The fibers are typically of a length of about 0.25 to 2 inches and typically about 1.2-15 denier.

The fibers may be laid in an overlapping, intersecting random arrangement to a thickness of about 0.25 inches or less to form a mat of fibers. The fibers may be arranged by most any convenient known manner such as by wet laying, air-laying or carding.

The fabric of the present invention is made by forming a mat of randomly arranged fibers. After the fibers are randomly arranged as desired, the binder compound is applied to the fibers. The latex binder is applied in an effective amount which will result in the fabric having sufficient strength and cohesiveness for the intended end use application. As well known in the art, the latex binder may be applied to the layer of randomly arranged fibers in a spaced, intermittent pattern of binder sites, or uniformly applied throughout the layer of fibers. The exact amount of the latex binder employed depends, in part, upon factors such as the type of fiber, weight of fibrous layer, nature of latex binder and the like. For example, end uses that require a stronger fabric may utilize more binder. A typical content of latex binder applied on a fiber mat (i.e. binder content) is about 15-45, preferably about 20-40, and more preferably about 25-35 wt %.

The latex is then cured using methods well known in the art such as by application of heat or radiation. The term “cured” refers to the latex being dried and a film being formed so as to increase the tensile strength properties of the non-woven as compared to the tensile strength of the non-woven absent any binder. The curing of the treated fibers is affected at a temperature above the glass transition temperature of the binder. It will be recognized that polymers may or may not include specific crosslinking agents for example NMA, NMMW, IBMA, epoxies, etc.

In order to demonstrate the practice of the present invention, the following examples have been prepared and tested. The examples should not, however, be viewed as limiting the scope of the invention. The claims will serve to define the invention.

### EXAMPLES

### Example 1

A batch reactor was initially charged with 73.4 parts by weight of the ionized water, 1.5 parts by weight of itaconic acid, an iron scavenger (EDTA), 4.2 parts by weight sodium dialkyl sulfosuccinate (a blend of about 90% by weight dihexyl and about 10% by weight dioctyl), and 3.5 parts by weight styrene.

About 0.45 parts by weight sodium persulfate and about 4.05 parts by weight deionized water were then added to the reactor. The reactor was then evacuated with a vacuum, purged with nitrogen, and maintained at about 80° C. for about 30 minutes.

About 37.6 parts by weight additional styrene, about 48.5 parts by weight butadiene, about 4.8 parts by weight acrylic acid, about 4 parts by weight methyl acrylic acid, and about 1 part by weight chain transfer agent were sequentially added to the reactor in various increments over the course of about 6 hours. The resulting polymer had a theoretical solids content of about 50.5% by weight, a theoretical glass transition temperature (Tg) of −17.9, an acid content of about 10.26 weight percent, and a gel content of about 73.2%.

The resulting latex binder was then applied to polyester fibers. After applying a latex binder to the non-woven fibers, the latex binder was oven dried at about 100° C. to bond the fibers and then cured at about 150° C. to form a dimensionally stable non-woven fabric.

### Example 2

A batch reactor was initially charged with 73.4 parts by weight of the ionized water, 1.5 parts by weight of itaconic acid, an iron scavenger (EDTA), 0.2 parts by weight of a 0.25 active sodium lauryl sulfate, and 3.5 parts by weight styrene.

About 0.45 parts by weight sodium persulfate and about 4.05 parts by weight deionized water were then added to the reactor. The reactor was then evacuated with a vacuum, purged with nitrogen, and maintained at about 80° C. for about 30 minutes.

About 37.6 parts by weight additional styrene, about 48.5 parts by weight butadiene, about 4.8 parts by weight acrylic acid, about 4 parts by weight methyl acrylic acid, and about 1.74 parts by weight of the 0.25 active sodium lauryl sulfate, and about 1 part by weight chain transfer agent were sequentially added to the reactor in various increments over the course of about 6 hours. The resulting polymer had a theoretical solids content of about 49.2% by weight, a theoretical glass transition temperature (Tg) of −17.9, an acid content of about 10.26 weight percent, and a gel content of about 80 to about 83%.

Various modifications and alterations that do not depart from the scope and spirit of this invention will become apparent to those skilled in the art. This invention is not to be duly limited to the illustrative embodiments set forth herein.

What is claimed is:

1. A binder composition for a non-woven fabric, the binder composition comprising:
a latex, the latex including a heterogeneous blend of dispersed polymer particles and a surfactant, where the particles include from about 55 to about 100% by weight of a gelled polymer, from about 1 to about 15% by weight polymeric units bearing an acid functionality, and where dried films of the latex exhibit a Tg of from about −50° C. to about 60° C.

2. The binder composition of claim 1, where the polymer particles includes from about 75 to about 98% by weight of a gelled polymer.

3. The binder composition of claim 1, where the polymer particles include from about 3 to about 12% by weight polymeric units bearing an acid functionality.

4. The binder composition of claim 1, where dried films of the latex exhibit a Tg of from about −35° C. to about 35° C.

5. The binder composition of claim 1, where the gelled polymer includes from about 15 to about 75% by weight units deriving from conjugated diene monomer.

6. The binder composition of claim 5, where the conjugated diene monomer is 1,3-butadiene.

7. The binder composition of claim 1, where the polymeric units bearing an acid functionality derived from acrylic acid, methacrylic acid, itaconic acid, or mixtures thereof.

8. The binder composition of claim 7, where the polymer particles include from about 0 to about 3% by weight units deriving from itaconic acid, from about 2 to about 7% by weight units deriving from acrylic acid, and from about 1 to about 6% by weight units deriving from methacrylic acid.

9. The binder composition of claim 7, where the polymer particles include from about 0 to about 8% by weight units deriving from acrylic acid, and from about 0 to about 8% by weight units deriving from methacrylic acid.

10. The binder composition of claim 7, where the polymer particles include from about 1 to about 7% by weight units deriving from acrylic acid, and from about 1 to about 7% by weight units deriving from methacrylic acid.

11. The binder composition of claim 7, where the polymer particles include from about 2 to about 6% by weight units deriving from acrylic acid, and from about 2 to about 6% by weight units deriving from methacrylic acid, and from about 0.5 to about 3% by weight units deriving from itaconic acid.

12. The binder composition of claim 7, where the polymer particles include from about 2.5 to about 5% by weight units deriving from acrylic acid, and from about 2.5 to about 5% by weight units deriving from methacrylic acid, and from about 1 to about 2% by weight units deriving from itaconic acid.

13. The binder composition of claim 1, where the latex is characterized by a pH of from about 4.5 to about 8.0.

14. The binder composition of claim 1, where the surfactant includes an alkali metal salt of an alkyl sulfosuccinate, a salt of alkyl sulfate, a salt of an organo disulfonate, or a mixture thereof.

15. The binder composition of claim 14, where the alkali metal salt of an alkyl sulfosuccinate includes alkali metal salts of mono and dialkyl sulfosuccinates, and where the alkyl substituents include from about 5 to about 12 carbon atoms.

16. The binder composition of claim 15, where the surfactant includes from about 50 to about 99% by weight sodium dihexyl sulfosuccinate compounds and from about 1 to about 50% by weight sodium dioctyl sulfosuccinate compounds.

17. The binder composition of claim 1, where the surfactant includes sodium dihexyl sulfosuccinate, sodium dioctyl sulfosuccinate, sodium octane sulfonate, an alkyl phenol ethoxylate, a fatty alcohol ethoxylate, an alkyl polyglucoside, an alkyl phosphate, sodium lauryl sulfate, sodium dodecyl diphenyloxide disulfonate, sodium laureth sulfate, Laureth-3, Laureth-4, Laureth-, Laureth-6, Laureth-7, sodium lauryl ether sulfate, sodium laureth-12 sulfate, sodium laureth-30 sulfate, or a mixture thereof.

18. The binder composition of claim 1, where the composition further includes a froth agent.

19. The binder composition of claim 18, where the froth agent includes disodium stearyl sulfosuccinamate.

20. The binder composition of claim 1, where the composition includes sodium laureth sulfate, and optionally an alkali metal salt of an alkyl sulfosuccinate, a salt of alkyl sulfate, or a salt of an organo disulfonate.

21. The binder composition of claim 1, where the composition includes sodium laureth sulfate and disodium stearyl sulfosuccinamate.

22. The binder composition of claim 1, where the composition includes sodium dodecyl diphenyloxide disulfonate and disodium stearyl sulfosuccinamate.

23. The binder composition of claim 1, where the composition includes an alkyl ether sulfate.

24. The binder composition of claim 23, where the composition further includes disodium stearyl sulfosuccinamate.

25. The binder composition of claim 1, where the latex composition includes from about 0.1 to about 10% by weight surfactant.

26. The binder composition of claim 1, where the polymer particles include from about 15 to about 75% by weight units deriving from styrene.

27. A non-woven fabric bound with a binder composition, the binder composition comprising:
a latex, the latex including a heterogeneous blend of dispersed polymer particles and a surfactant, where the particles include from about 55 to about 100% by weight of a gelled polymer, from about 1 to about 15% by weight polymeric units bearing an acid functionality, and where dried films of the latex exhibit a Tg of from about −50° C. to about 60° C.

28. The non-woven fabric bound with a binder composition of claim 27, where the polymer particles include from about 75 to about 98% by weight of a gelled polymer.

29. The non-woven fabric bound with a binder composition of claim 27, where the polymer particles include from about 3 to about 12% by weight polymeric units bearing an acid functionality.

30. The non-woven fabric bound with a binder composition of claim 27, where dried films of the latex exhibit a Tg of from about −35° C. to about 35° C.

31. A diaper sub-layer comprising a non-woven fabric bound with a binder composition, the binder composition comprising:
a latex, the latex including a heterogeneous blend of dispersed polymer particles and a surfactant, where the particles include from about 55 to about 100% by weight of a gelled polymer, from about 1 to about 15% by weight polymeric units bearing an acid functionality, and where dried films of the latex exhibit a Tg of from about −50° C. to about 60° C.

32. The diaper sub-layer of claim 31, where the polymer particles includes from about 75 to about 98% by weight of a gelled polymer.

33. The diaper sub-layer of claim 31, where dried films of the latex exhibit a Tg of from about −35° C. to about 35° C.
